(19) United States
(12) Patent Application Publication
(10) Pub. No.: US 2023/0135198 A1
(43) Pub. Date: May 4, 2023

(54) SELF-SERVICE STATION HAVING THERMAL IMAGING CAMERA

(71) Applicant: Elenium Automation Pty Ltd, Tullamarrine (AU)

(72) Inventors: Nicholas Peter OSBORNE, Doreen (AU); Aaron Jason HORNLIMANN, Melbourne (AU)

(73) Assignee: Elenium Automation Pty Ltd, Tullamarine (AU)

(21) Appl. No.: 17/912,355

(22) PCT Filed: Mar. 17, 2021
(86) PCT No.: PCT/AU2021/050239

§ 371 Date: Sep. 16, 2022

(30) Foreign Application Priority Data
Mar. 17, 2020 (AU) 2020900817

(51) Int. Cl. G06Q 20/40; G06V 40/10; G06V 40/16; G06Q 20/18; G01J 5/00

(52) U.S. Cl. CPC G06Q 20/40145; G06V 40/15; G06V 40/161; G06Q 20/18; G01J 5/0025; H04N 23/11

(57) ABSTRACT

Embodiments relate generally to systems, methods, and processes that use thermal imaging at self-service interaction stations. In particular, embodiments may relate to a self-service station for conducting an interaction process, having a thermal imaging device configured to capture thermal images of an area, a processor configured to process the thermal images captured by the thermal imaging device, determine a temperature condition of a face identified in at least one of the captured thermal images and suspend the interaction process based on the determined temperature condition.

### CROSS-REFERENCE TO RELATED APPLICATIONS

The present application claims priority from Australian Provisional Patent Application No 2020900817 filed on 17 Mar. 2020, the contents of which are incorporated herein by reference in their entirety.

### TECHNICAL FIELD

Embodiments relate generally to systems, methods, and processes that may use thermal imaging at self-service interaction stations.

### BACKGROUND

As air travel becomes more affordable, there are greater numbers of passengers passing through airports in order to reach their destinations. Airlines and airports offer self-service channels in order to improve the customer experience and passenger processing volume capabilities with customer convenience and more efficient use of space in an increasingly busy airport environment. As a consequence of increased people movement across borders, airports, airlines and immigration departments are acutely aware of the increased potential for transmission of contagious illness to other passengers in an airport or aircraft, as well as to other people in the country of travel or destination. To reduce risk of transmission of contagious illnesses, various forms of early symptom detection have been employed in an attempt to filter out and manage travelling persons who may be ill and able to transmit the illness to other persons in the same vicinity.

One of these systems used is the implementation of an infrared thermal imaging camera at staffed touchpoints where the camera is focused at face-height of a person and the staff member checks for thermal data within the symptomatic range of an illness. This process requires the presence of a trained staff member at every staffed touchpoint in order to view the images captured by the camera to check every passenger for a positive or negative finding, which can be time consuming.

In situations where detection of illness symptoms must be applied more acutely, such as in the case of an epidemic or pandemic, most airport and airlines operations teams will be forced to close self-service channels to ensure that all persons must be processed by staff at a staffed counter. This causes significant operational impact to traffic flow which negatively impacts downstream operations. The negative impact can include a negative commercial impact to the airport duty free shopping, on-time performance of flights, and so on.

It is desired to address or ameliorate one or more shortcomings or disadvantages associated with prior techniques for thermal imaging and illness identification of airport or other transit customers or passengers, or to at least provide a useful alternative thereto.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

Some embodiments relate to a self-service station for conducting an interaction process, comprising: a memory storing executable program code, a processor in communication with the memory, the processor configured to execute the program code stored within the memory; a user interface in communication with the processor, the user interface configured to allow a user to initiate the interaction process; a thermal imaging device in communication with the processor, the thermal imaging device configured to capture thermal images of an area from which the user interface is accessible; wherein the processor is configured to: process thermal images captured by the thermal imaging device; determine a temperature condition of a face identified in at least one of the captured thermal images; and suspend the interaction process based on the determined temperature condition.

The processor may be configured to identify front-of-face thermal data based on the thermal images.

The station may further comprise a data store, wherein the processor is further configured to compare temperature data derived from the thermal images to illness-related temperature data stored in the data store and to suspend the interaction process when the temperature data matches illness-related temperature data. The data store may comprise a repository of temperature data profiles associated with symptoms of at least one illness.

The interaction process may comprise a multi-step process that can take between about 1 minute and about 20 minutes to complete when not suspended.

The processor may be configured to determine heart rate data based on changes over time in images of the face identified in the at least one of the thermal images.

The station may further comprise a colour camera, positioned to capture RGB images of the area simultaneously with capture of the thermal images.

The processor may be configured to receive captured RGB images from the colour camera and to determine heart rate data based on changes over time in images of the face identified in the captured RGB images. In some embodiments, the processor is configured to suspend the interaction process based on the determined temperature condition and the determined heart rate data.

The processor may be configured to compare the determined heart rate data to stored illness-related hear rate data accessible to the processor.

The processor may be configured to generate an alert based on the determined temperature condition. In some embodiments, the processor is configured to send the alert via a communication module of the station to a server or a client device over a network that is accessible to the communication module. The alert may include a unique identifier of the station and at least one of: an image of the face; the determined temperature condition; user identification information received via the user interface; illness-related information associated with the determined temperature condition; or user booking information retrieved from a data store based on user identification information received via the user interface.

The processor may be configured to use a trained machine learning model to identify the face in at least one of the captured thermal images.

The processor may be configured to identify multiple faces in at least one of the captured thermal images and to determine which of one or more of the multiple faces are proximate to the station.

The processor may be configured to determine the temperature condition of each of the multiple faces and to suspend the interaction process when the temperature condition of at least one of the multiple faces matches a temperature data profile associated with symptoms of at least one illness.

Some embodiments relate to a system for conducting self-service interaction processes, the system including: at least one self-service station according, and at least one client computing device in communication with the at least one self-service station;

The processor of each of the at least one self-service station may be configured to resume the interaction process in response to receiving a resume message from the at least one client computing device.

The system may further comprise a server, in communication with the at least one self-service station and the at least one client computing device. Communication between the at least one self-service station and the at least one client computing device may be routed through the server.

Some embodiments relate to a computer-implemented method of conducting an interaction process at a self-service station comprising the steps of: receiving an initiation request at a user interface to initiate an interaction process; capturing a thermal image with a thermal imaging device; processing a thermal image to determining whether temperature data based on the thermal image matches a stored profile; and suspending the transaction process when it is determined that the temperature data matches a stored profile.

Some embodiments relate to a system for conducting an interaction process comprising: at least one of the self-service station, wherein the at least one self service station is configured to transmit alert data over a network if the interaction process is suspended; and a client device configured to receive alert data from the self-service station over the network. The client device may be configured to send a message to the station to cancel or resume the interaction process.

The station may further comprise a proximity sensor in communication with the processor, the proximity sensor being configured to determine a proximity measurement in the area from which the user interface is accessible. The processor may be further configured to suspend the interaction process based on the determined proximity measurement.

The processor may be further configured to determine a respiratory rate based on thermal images of the face identified in captured thermal images received over a time interval. The processor may be further configured to suspend the interaction process based on the determined respiratory rate.

### BRIEF DESCRIPTION OF DRAWINGS

FIG. 1 is a block diagram view of an interaction station system according to some embodiments;

FIG. 2 is a block diagram view of an interaction station network according to some embodiments;

FIG. 3 is a block diagram view of a user at an interaction station according to some embodiments;

FIG. 4 is a field of view of a thermal imaging device according to some embodiments;

FIG. 5 is a first flow chart of the operation of the interaction station according to some embodiments;

FIG. 6 is a second flow chart of the operation of the interaction station according to some embodiments;

FIG. 7 is a schematic illustration of a temperature heuristic used in symptom matching according to some embodiments;

FIG. 8 is a flowchart of a further method of operation of the interaction station according to some embodiments; and

FIG. 9 is a schematic block diagram of a computer system architecture that can be employed according to some embodiments.

### DETAILED DESCRIPTION

Embodiments relate generally to systems, methods, and processes that may use thermal imaging at self-service interaction stations.

In some embodiments, a self-service interaction station 101 is provided to facilitate users conducting interaction processes. The stations 101 are connected to a client device 145 and database 155 over a network 140. The station 101 is configured to analyse Front-of-Face (FoF) temperature data of persons within the field of view of a thermal imaging device 125. The station 101 is further configured to selectively suspend the interaction process based on matches between FoF data and stored thermal profiles relating to at least one illness. The stored thermal profiles may comprise temperature conditions related to at least one illness.

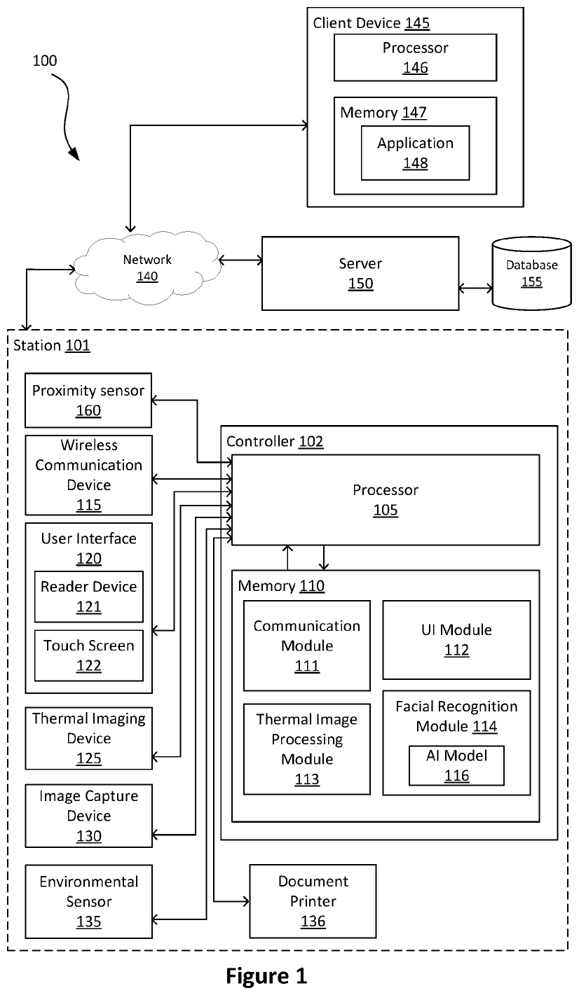

FIG. 1 is a block diagram of a system 100 for managing self-service interaction stations, comprising a station 101, a server 150, a database 155 accessible to the server 150, and at least client device 145. Station 101 is in communication with server 150 and client device 145 over a network 140.

In the embodiments of FIG. 1, station 101 may comprise a controller 102. The controller 102 comprises a processor 105 in communication with a memory 110 and arranged to retrieve data from the memory 110 and execute program code stored within the memory 110. Station 101 may be connected to network 140, and in communication with client device 145, server 150, and database 155.

Processor 105 may include more than one electronic processing device and additional processing circuitry. Processor 105 may execute all processing functions described herein locally on the station 101 or may execute some processing functions locally and outsource other processing functions to another processing system, such as server 150. Processor 105 may include multiple processing chips, a digital signal processor (DSP), analog-to digital or digital-to analog conversion circuitry, or other circuitry or processing chips that have processing capability to perform the functions described herein.

The network 140 may comprise at least a portion of one or more networks having one or more nodes that transmit, receive, forward, generate, buffer, store, route, switch, process, or a combination thereof, etc. one or more messages, packets, signals, some combination thereof, or so forth. The network 140 may include, for example, one or more of: a wireless network, a wired network, an internet, an intranet, a public network, a packet-switched network, a circuit-switched network, an ad hoc network, an infrastructure network, a public-switched telephone network (PSTN), a cable network, a cellular network, a satellite network, a fiber optic network, some combination thereof, or so forth.

Server 150 may comprise one or more computing devices configured to share data or resources among multiple network devices. Server 150 may comprise a physical server, virtual server, or one or more physical or virtual servers in combination.

Database 155 may comprise a data store configured to store data from network devices over network 140. Database 155 may comprise a virtual data store in a memory of a computing device, connected to network 140 by server 150.

Station 101 may further comprise a wireless communication device 115, user interface 120, thermal imaging device 125, image capture device 130, environmental sensor 135, and document printer 136. Station 101 may further comprise proximity sensor 160. The proximity sensor 160 may be housed within housing 315. Proximity sensor 160 may be configured to determine a proximity measurement in the area from which the user interface is accessible.

Wireless communication device 115 may comprise a wireless Ethernet interface, SIM card module, Bluetooth connection, or other appropriate wireless adapter allowing wireless communication over network 140. Wireless communication device 115 may be configured to facilitate communication with external devices such as client device 145 and server 150. In some embodiments, a wired communication means is used.

User interface 120 may comprise a touchscreen 122, keyboard, or other device allowing a user of the station initiate and interact with an interaction process. The user interface 120 may further comprise a reader device 121 configured to allow a user to initiate and interact with an interaction process. In some embodiments, the interaction process comprises a series of steps allowing a user 305 to provide identification details to the station 101 to retrieve booking details and/or undertake a check-in process. In some embodiments, the interaction process may comprise a series of steps wherein the user 305 provides booking details to the station 101 to identify themselves. In some embodiments, the interaction process may take between 1 and 20 minutes, for example. In other embodiments, the interaction process may take other appropriate ranges of time, allowing the user sufficient time to undertake the interaction process, and have thermal images of the image capture area 310 captured and processed by the station 101.

The reader device 121 may comprise a barcode scanner, QR code scanner, magnetic strip reader, or other appropriate device arranged to allow a user to scan a document (such as a passport, boarding pass, ticket, or other identification document) at the station 101. In such embodiments, the data read by the reader device 122 may be stored in the memory 110, or transmitted to database 155 through the server 150 over a network 140. In other embodiments, the data read by the reader device 121 may trigger the processor 105 to send a request for information associated with the data over network 140 to the server 150. The server 150 may then retrieve additional data associated with the identification data form database 155 and transmit the additional data over network 140 to the processor 105.

Thermal imaging device 125 may comprise a thermal camera, arranged to capture thermal image frames of people within an area from which the user interface 120 is accessible. In some embodiments, thermal imaging device 125 comprises an infrared thermal imaging camera (ITIC), capable of capturing infrared thermal image data from a field of view of the camera. The thermal imaging device may output a thermal image comprising a pixel colour map, the colour defining the detected thermal temperature of the object in that pixel. The device 125 may provide a reference for colour to temperature value, thereby allowing the thermal image processing module 113 to ascertain the temperature value of a given pixel.

Thermal imaging device 125 may transmit thermal image frames to memory 110 for processing by the thermal image processing module 113. In some embodiments, thermal imaging device 125 has image processing capabilities, and conduct an initial processing stage before transmitting the image to memory 110.

Thermal imaging device 125 may comprise a thermal camera capable of meeting standard requirements for screening thermographs for human febrile temperature screening, such as such as IEC 80601-2-59 (2017-09), and/or other ISO/IEC requirements. Thermal imaging device 125 may be able to detect temperature increments with high accuracy, such as a temperature increment less than 0.1° C. and greater than 0, for example. The temperature increment may be less than 0.1° C. and greater than 0.01° C., for example. The thermal imaging device 125 may be configured to output thermal image frames of a large or high enough resolution to accurately identify facial regions of a person within a frame. In some embodiments, the resolution has a minimum size of 320×240 pixels, and the thermal imaging device 125 is positioned so that the face of a user may fill at least 180×240 pixels (or about 56% of the total image size). In some embodiments, the thermal imaging device 125 may be positioned so that face of a user may fill about 50% of the total image size of the thermal image. In other embodiments, the thermal imaging device 125 may be positioned so that a face of a user may fill no less than 40% of the total image size of the thermal image. In other embodiments the thermal imaging device 125 may be positioned so that a face of a user fills no less than 30%, 25%, or 20% of the thermal image. The size of a user's face in the thermal image may be sufficient to allow clear differentiation of facial regions, to better enable symptom matching, tracking of respiratory rate, and other thermal analysis.

In some embodiments, the thermal imaging device 125 may comprise a thermal camera, such as the Seek Thermal™ Mosaic Core 320×240 model for example.

Image capture device 130 may comprise a camera, arranged to capture images of an area from which the user interface 120 is accessible. In some embodiments, image capture device 130 comprises a digital camera device.

Proximity sensor 160 may be in communication with processor 105, and comprise an infrared proximity sensor configured to determine a distance between the proximity sensor 160 and a person or object in an area in front of the station 101 (from where the person can access user interface 120). In other words, the proximity sensor faces the same way as the thermal imaging device 125 and the image capture device 130. The output of the proximity sensor may be sent to controller 102, where a determination may be made as to whether a person or object is within a minimum or maximum distance from the thermal imaging device 125 and/or the image capture device 130.

Environmental sensor 135 may comprise a temperature sensor, arranged to detect ambient temperature in the vicinity in and around the station 101. In some embodiments, the environmental sensor also detects humidity, pollutant levels, or other environmental effects. Data from the environmental sensor may be sent to thermal image processing module 113 in memory 110, in order to provide a baseline environmental reading. This baseline reading may be compared with thermal images captured by thermal imaging device 125, or used in symptom matching.

Document printer 136 may comprise a printer configured to allow for printing user documents as a result of the interaction process. In some embodiments, the document printer 136 prints boarding passes, receipts, or other documentation related to the user or the interaction process.

The memory 110 may further comprise executable program code that defines a communication module 111, user interface (UI) module 112, thermal image processing module 113, and facial recognition module 114. The memory 110 is arranged to store program code relating to the communication of data from memory 110 over the network 140.

Communication module 111 may comprise program code, which when executed by the processor 105, implements instructions related to initiating the wireless communication device 115. When initiated by the communication module 111, the wireless communication device 115 may send or receive data over network 140. Communication module 111 may be configured to package and transmit data generated by the UI module 112 and/or the thermal image processing module 113 and/or retrieved from the memory 110 over network 140 to a client device 145, and/or to server 150. In some embodiments, this transmitted data includes an alert, relating to a person identified by thermal imaging device 125 or image capture device 130. In some embodiments, the alert relates to images processed by thermal image processing module 113. In some embodiments, the alert relates to data captured by touch screen 122 or reader device 121.

UI module 112 may comprise program code, which when executed by the processor 105, implements instructions relating to the operation of user interface 120. The memory 110 may further comprise a thermal image processing module 113, arranged to store program code relating to the operation of the thermal imaging device 125.

Thermal image processing module 113 may comprise program code, which when executed by the processor 105, implements instructions configured to allow the module 113 to receive captured thermal image frames from the thermal imaging device 125. The thermal image processing module 113 may be configured to process thermal image frames from the thermal imaging device 125. In some embodiments, this process compares the captured thermal images from the thermal imaging device 125 to thermal profiles relating to symptoms of at least one illness. In some embodiments, the thermal profiles comprise illness-related temperature data. Thermal image processing module 113 may be further configured to issue instructions to the processor 105 relating to the operation of the station 101 based on the processed thermal images.

In some embodiments, thermal image processing module 113 may further comprise Front-of-Face (FoF) recognition algorithms. The algorithms configured to analyse captured thermal image frames to locate human FoF areas within the image capture area. In such embodiments, the thermal image processing module 113 compares the FoF data against thermal image profiles associated with symptoms of at least one illness. In some embodiments, the thermal image profiles are stored within database 155. In other embodiments the thermal image profiles are stored within the thermal image processing module 113, or memory 110.

In some embodiments, thermal image processing module 113 may further process the image outputs from the thermal imaging device 125 and image capture device 130 using facial feature recognition techniques as described herein for the purpose of identifying the location of the inner or medial canthus (tear duct) region of a person within the image frame. Using the medial canthus region of the face of a user for image-based temperature determination may allow for improved accuracy of body temperature detection over temperatures determined from the forehead area, for example.

In such embodiments, the thermal image processing module 113 compares the thermal data collected from the pixels of the medial canthus region in the image frame against the thermal profiles associated with symptoms of at least one illness. In some embodiments, the thermal image processing module 113 may invoke functions of the facial recognition module 114 to identify pixels in captured images corresponding to the medial canthus area. Identifying such pixels may include detecting an eye location of a user, and calculating the distance from a centroid or another part of the eye location to the canthus of the eye, in order to assist with the accuracy of identification of the medial canthus and thus improve accuracy of the temperature measurement.

In some embodiments, the thermal image processing module 113 may further comprise program instructions to perform respiratory rate detection processes. The processes analyse captured thermal image frames to isolate a pixel region around a facial region that changes temperature during respiration cycles, for example such as a person's nose, and track the thermal data of those pixels of the pixel region over a series of image frames. The thermal image processing module 113 may then determine a respiratory rate based on the change in temperature of the pixels over time. For example, the respiratory rate may be determined based on a time period elapsed between a time of maximum detected temperature of one or multiple pixels in the pixel region and a time of minimum detected temperature of the one or more pixels. In an example where the facial region includes the nose, a higher temperature on an upper lip surface beneath the nostrils may be associated with exhalation of a person through their nose. Conversely, a lower temperature on an upper lip surface beneath the nostrils may be associated with inhalation of a person through their nose. In some embodiments, the pixel region may include the nostrils and/or the region immediately below the nostrils of a person and/or in the philtrum area above the vermillion border.

The respiratory rate may be determined in breaths per minute, for example. The determination of respiratory rate may be undertaken over a time interval, such as one minute, for example. The time interval may be less than a minute but more than 10 seconds, in some embodiments. In other embodiments, the time interval may be more than a minute and less than 5 minutes, or up to an end time of the user interaction, for example. In other embodiments, other time intervals may be used to determine respiratory rate. For example, a multiple (such as 2, 3, 4, 5, 6, 7, 8, 9, 10, 11 or 12) of an average time of an expiration and inhalation cycle may be used as a lower bound for the respiratory rate determination time interval. In another example, the respiratory rate may be determined repeatedly over the entire period of the interaction in order to determine a maximum respiratory rate and/or to determine patterns in respiratory rate. The assessed outcome of the respiratory rate determination process may be processed by the thermal image recognition module 113 for symptom match and may be used to determine whether the user interaction should be suspended or resumed, and/or an alert sent to the station operator.

In some embodiments, the determination of the respiratory rate may be based on the maximum temperature readings of the pixel region over a time interval. The maximum temperature readings may comprise peak temperatures corresponding to the exhalation of breath.

In some embodiments, the determination of the respiratory rate may be based on the maximum and minimum temperature readings of the pixel region over a time interval. The maximum temperature regions may comprise high peak temperatures corresponding to exhalation of breath, and the minimum temperature readings may comprise low peak temperatures corresponding to the inhalation of breath. In other embodiments, the determination of the respiratory rate may be based on the minimum temperature readings of the pixel region over a time interval.

Facial recognition module 114 may comprise executable program code (instructions), which when executed by the processor 105, implements instructions configured to allow the module 114 to identify pixel regions in images that correspond to faces within the image frame. Facial recognition module 114 may further comprise an artificial-intelligence (AI) model 116, trained on facial image frames. AI model 116 may be trained using supervised machine learning in order to accurately provide instructions to facial recognition module 116 to identify faces in image frames. In some embodiments, images captured by thermal imaging device 125 or image capture device 130 are stored in memory 110, or facial recognition module 114 for verification by a human operator. The human verification of the stored image frames as containing a face or not may be used to generate the AI model 116. AI model 116 may utilise machine learning algorithms and increase accuracy of face detection by facial recognition model 114.

In some embodiments, the station 101 may operate in an AI model 116 training mode in order to generate an accurate model of for automatic face detection, developed specific to an individual station 101. In such embodiments, the individual generation of AI model 116 can accommodate the particular location, positioning, angle, and lighting of the image capture area 310 of a particular station 101.

In some embodiments, the AI model 116 may be pre-generated (i.e. previously trained) with known face detection algorithms and image frames from a data store, such as database 155 or memory 148.

The AI model 116 may be trained on a data set of captured images from the image capture device 130, a pre-existing data set of images from other sources, or some combination thereof. The AI model 116 may be supervised, through manual review of control images wherein the AI model 116 identifies a face. The AI model 116 may be trained on a dataset that includes multiple faces in an image, occluded faces, blurry images, or images where the colour and rotation make face detection more difficult, for example, in order to improve the accuracy of face detection in use. The difficulty of the selected dataset may allow the model to correctly classify partially occluded faces (such as when a person is wearing a face mask).

The AI model 116 may use captured images from the image capture device 130 to continually develop a more accurate model throughout its normal operation.

AI model 116 may comprise one or more software processes configured to estimate bounding boxes of a face within an image without prior scale and position. AI model 116 may comprise one or more software processes configured to detect the presence of a face in an image through face localisation techniques such as, face alignment, pixel-wise face parsing, and 3D dense correspondence regression, for example. AI model 116 may comprise a single stage face identification software process, for example. In some embodiments, a commercially available or published face recognition model or framework may be used to build AI model 116 in a way that uses facial landmark recognition, such as RetinaFace (“RetinaFace: Single-stage Dense Face Localisation in the Wild”, Deng et al, 4 May 2019), for example. In such embodiments, the considerations for the model choice may involve the ability to consistently locate faces in images across a wide variety of contexts and conditions.

Client device 145 may comprise a smartphone, tablet computing device, personal computer, or other appropriate device configured to receive and transmit data over a network. Client device 145 may further comprise a processor 146 in communication with a memory 147. The processor 146 is configured to access or modify executable instructions within memory 147. Memory 147 may further comprise a special purpose application 148, often generically called an “app”. The application 148 may comprise executable program code, which when executed by the processor 148 allows the client device 145 to interact with station 101, and/or server 150 over network 140. An operator of the client device 145 may interact with the application 148 to cause the client device 145 to communicate with the station 101 to read or modify data of a user of the station 101, to pause, resume, cancel, or initiate an interaction process at station 101.

In some embodiments, an operator of client device 145 may initiate a request from the application 148 to cause the client device 145 to issue commands to a station 101. The request is forwarded by processor 146 over network 140 to station 101. The request is then sent by the wireless communication device 115 to controller 102. The processor 105 may then process the request, and selectively access or modify memory 110 as instructed. In some embodiments, this request is to access or modify user data stored in memory 110. In other embodiments, this request is to access or modify data stored in communication module 111, UI module 112, or thermal image processing module 113. In other embodiments, the request provides instructions to processor 105 to activate, deactivate, or interact with wireless communication device 115, user interface 120, thermal imaging device 125, image capture device 130, environmental sensor 135, or document printer 136.

In some embodiments, an operator of client device 145 initiates a request from the application 148 to cause the client device 145 to issue commands to the database 155. The request is forwarded by processor 146 over network 140 to server 150. Server 150 may then access or modify the data stored within database 155. In some embodiments, this data comprises user data records relating to interaction processes conducted at station 101, thermal image profiles associated with symptoms of at least one illness, airline and passenger data, or other types of data.

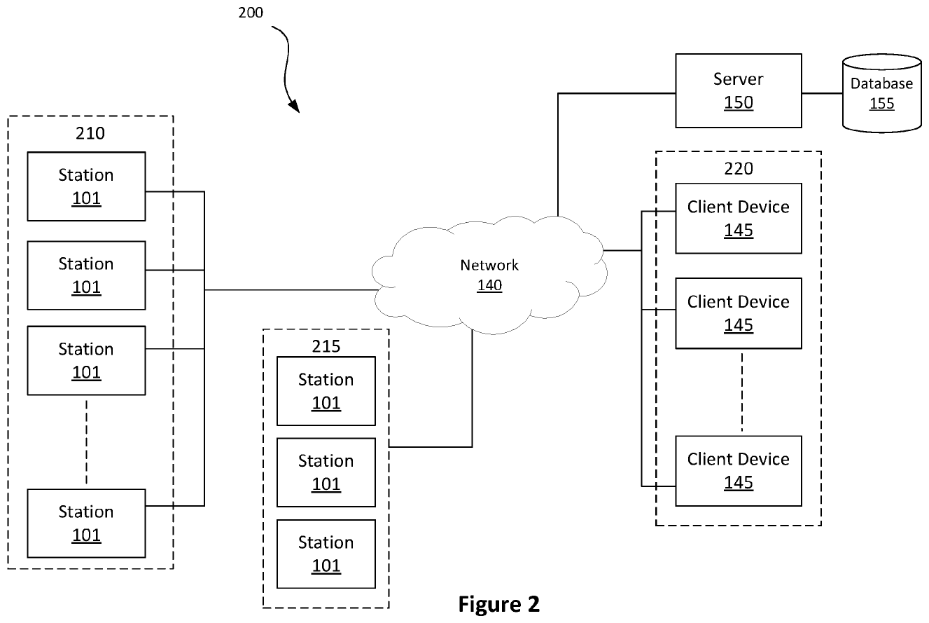

FIG. 2 depicts a block diagram of a self-service station network 200 according to some embodiments. The network 200 comprises an individual self-service station bank or array 210, a separately located self-service station bank or array 215, server 150, database 155, and client device array 220. The individual self-service station array 210 may comprise at least one self-service station 101 individually connected to network 140. In some embodiments, the stations 101 of array 210 are located together at a single installation site, such as an airport check-in, or an airport immigration area. In other embodiments, the stations 101 of array 210 may be separately located throughout a number of individual sites throughout an airport, or may be located at multiple installation sites, such as a series of airports. In some embodiments, the locations of installation of array 210 comprise self-service facilities including, but not limited to, self-service check-in kiosks, self-service bag drop, automated departure gate boarding gates, automated immigration entry or exit gates, airline lounge gates, or other appropriate self-service areas, for example.

The client device array 220 may comprise at least one client device 145 connected individually to network 140. In some embodiments, the array 220 comprises any combination of smartphones, tablet computing devices, personal computers, or other devices capable of sending instructions over network 140 and executing instructions from memory 147.

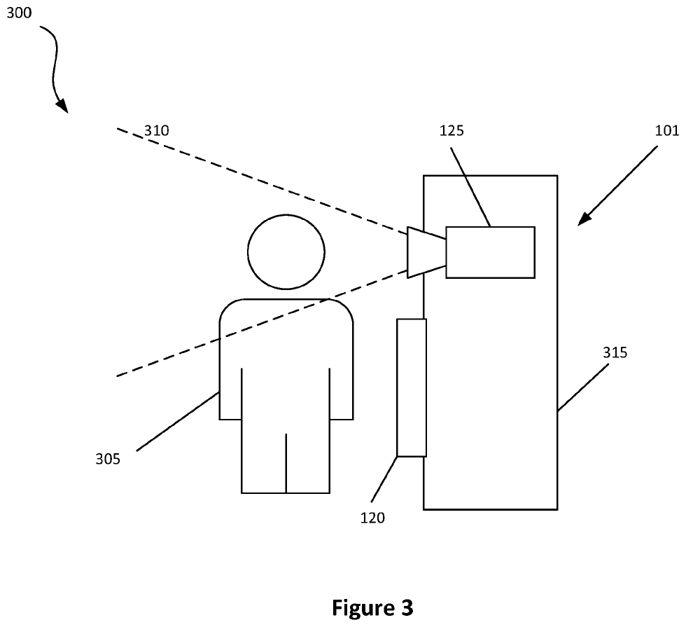

FIG. 3 depicts a diagram 300 of image capture of a user 305 interacting with a self-service station 101. The self service station 101 further comprises a housing 315. The housing 315 houses the components of the self-service station 101 as described herein. The housing 315 may entirely enclose the components of the self-service station 101, except for user interface 120 and except to allow images and sensor readings to be captured. In the pictured embodiment, user 305 is at least partially within the image capture area 310 of thermal imaging device 125. The thermal imaging device 125 is positioned to capture images in an area from which the user interface 120 is accessible. The thermal imaging device 125 may be positioned to ensure the image capture area 310 defines an area substantially facing the direction from which the user interface may be accessed from a user 305. In some embodiments, the user 305 may be an airline passenger, airline or airport staff, or other individual at an airport requiring self-service interaction or check-in processes. In some embodiments, the user 305 may be a train, ship or other transport passenger, staff, or other individual requiring self-service interaction or check-in processes for transport purposes. In some embodiments, the user 305 may be an event participant, attendee at a secure facility or other person requiring self-service check-in processes.

In some embodiments, the image capture area 310 defines a horizontal range of approximately 1 meter either side of the anticipated position of a user 305 using the user interface 120. In some embodiments, the image capture area 310 defines a vertical range of about 0.5 meters above and below the anticipated position of a user 305 using the interface 120.

In some embodiments, the image capture area 310 is substantially centred at an anticipated average height of an adult person who would be accessing the user interface 120. The image capture area 310 may extend in a horizontal and vertical area to cover other people close to the user 305. In some embodiments, other appropriate ranges may be defined. In other embodiments, the image capture area 310 may be arranged to be substantially centred at the anticipated area of the upper portions of a user 305. The upper portions of a user 305 are intended to include at least the user's chest, neck, face, and head.

In other embodiments, the image capture area 310 may be dynamically altered by the thermal imaging device 125 to be extended, shrunk or laterally or vertically shifted in accordance with specified requirements.

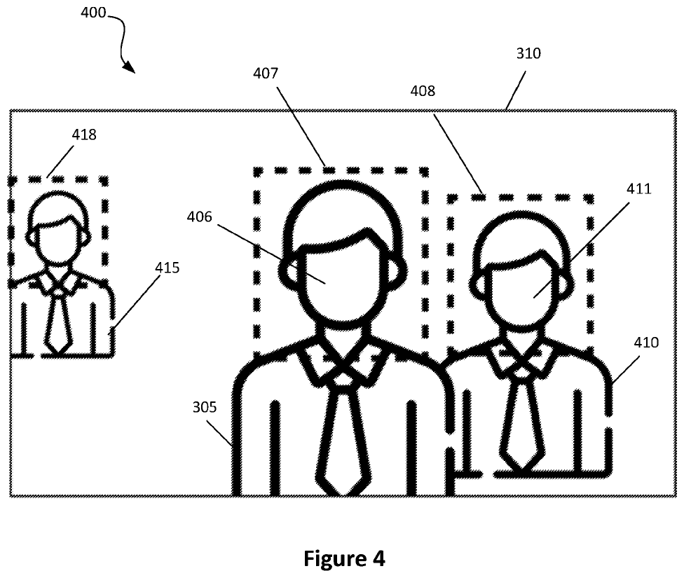

FIG. 4 depicts a an example of a thermal image frame 400, depicting a user 305, a second person of interest 410, and a third person of interest 418, with identified facial regions 407, 408, and 418 within the image capture area 310.

In some embodiments, the illustration of FIG. 4 comprises a thermal image 400 captured by the thermal imaging device 125. In such embodiments, the thermal image 400 comprises a pixel colour coded heat map of the image capture area, wherein each pixel is assigned a colour based on its temperature. In such embodiments the thermal image processing module 113 is be configured to analyse the thermal image based on an algorithmic model to detect the FoF area of people within the image capture area 310. In some embodiments, these people comprise the user 305, second person 410, and third person 418. In some embodiments, the algorithmic model comprises a machine learning-based model trained to detect the FoF and head area of any person or persons captured within the image capture area 310. In some embodiments, the algorithmic model is AI model 116 within facial recognition module 114.

The thermal image processing module 113 may further isolate the identified FoF and head areas into identified facial regions 407, 408, and 418. Thermal image processing module 113 may then generate specific FoF frame data for further processing based on the isolated regions.

In some embodiments, this further processing comprises a proximity analysis. In such embodiments, the thermal image processing module 113 isolates facial regions from the thermal image frame 400, to produce an isolated facial region frame. Thermal image processing module 113 may further compare the isolated facial region frames against proximity threshold levels to determine whether a FoF frame corresponds to a user, a person adjacent or proximally close to a user, or a person distant from the user. In some embodiments, face proximity threshold levels are specified or determined by facial recognition module 114.

In some embodiments, the thermal image processing module 113 is configured to evaluate pixel size of the FoF area to determine whether a FoF frame should be retained for symptom analysis. In such embodiments, if an identified face does not meet a threshold size requirement the module 113 may deem the face too far for accurate processing and discard the frame associated with it. In such embodiments, the person 415 with detected facial region 418 is deemed too far away. In some embodiments, face size threshold requirements are specified or determined by facial recognition module 114.

In some embodiments, identification of whether a person is within an acceptable distance from the station 101 may further include the use of proximity sensor 160. Proximity sensor 160 may be configured to take a distance measurement between a person and a front side of the station 101 (from which the user interface 120 is accessible). Based on output signals received from the proximity sensor 160, the controller 102 controls the user interface 120 to selectively allow or prohibit interaction with the station 101 if a person is within a minimum distance, or outside a maximum distance, respectively. The output of proximity sensor 160, when processed by controller 102, may trigger an alert from the UI module 112 to be displayed on user interface 120 if a person does not meet the distance requirement, suspending or pausing the operation of a transaction on the station 101 until a person stands within the distance requirement. In some embodiments, the distance requirement may be a minimum distance of up to 1 meter, or 0.5 meters for example. In some embodiments, the distance requirement may be a maximum distance requirement, requiring a user to stand no closer than 1 meter, or 0.5 meters for example. In some embodiments, a combination of minimum distance and maximum distance requirements may be employed by controller 102 to enable the user interaction to proceed via the user interface 120. In other embodiments, other ranges may be used. The distance requirements may be specified in order to assist in directing a user of the station 101 to stand within the field of view of image capture device 130 or thermal imaging device 125.

In some embodiments, the size threshold levels of a facial region 408 are compared against proximity to the largest identified facial region 407. In such examples it may be beneficial to identify and retain the FoF data of those near or with the user 305. Persons identified within this threshold may correspond to friends, family, or travelling companions of the user 305. Accordingly it may be beneficial to conduct thermal analysis on such persons within the image capture area 310.

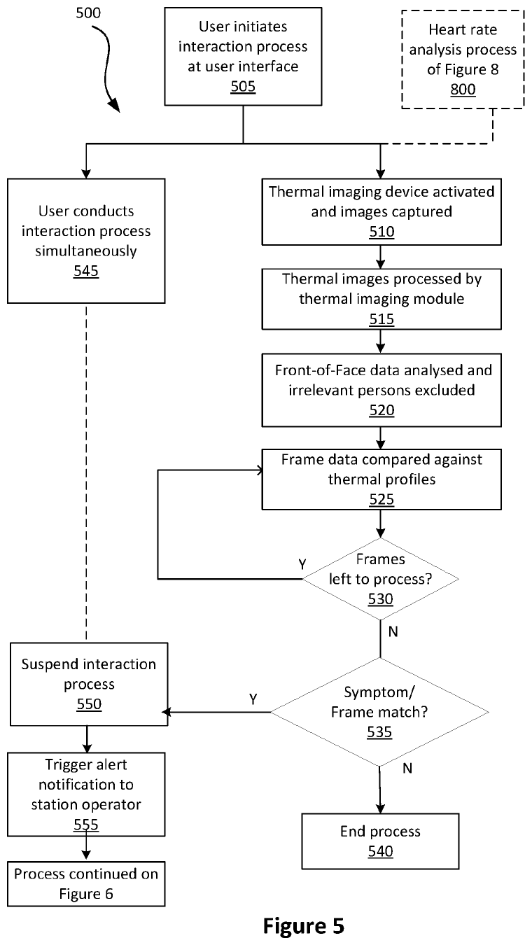

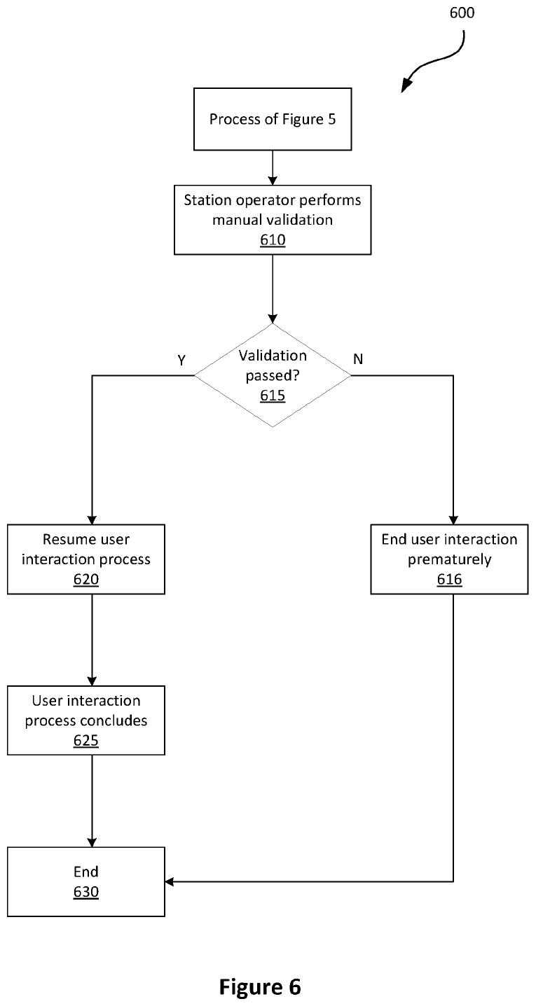

FIG. 5 and FIG. 6 are flow charts of example embodiments of a process 500 executed by a self-service station 101.

In such embodiments, a user approaches the self-service station 101 and initiates an interaction process at a user interface 120 and/or touch screen 122, at step 505. In some embodiments, this involves following the instructions presented on the touch screen 122 in order to retrieve a booking from memory 110 to begin the interaction process.

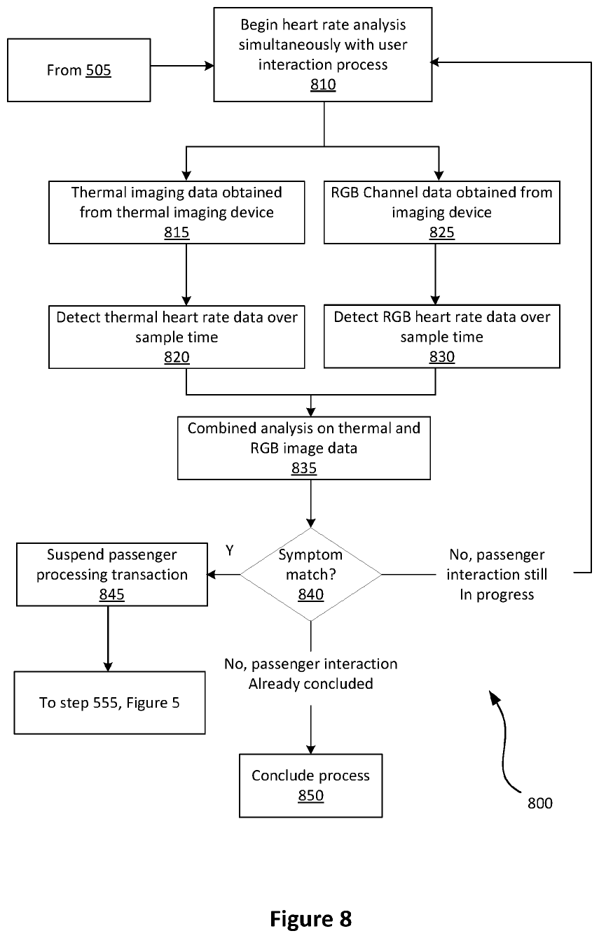

Once the interaction process has begun the user 305 may be directed by a series of on-screen instructions on touch screen 122 at step 545, while the parallel task of the detection and possible alerting of illness symptoms is executed concurrently by the thermal image processing module 113. In some embodiments, the heart rate monitoring process 800 (described further below in relation to FIG. 8) may be initiated in parallel at this step, in order to determine the heart rate of a person within the image capture area 310.

At the beginning of the simultaneous thermal imaging process, the thermal imaging device 125 begins capturing thermal image frames at 510. The image frames containing the thermal data of objects observed within the image capture area 310. In some embodiments, image capture device 130 obtains standard colour (red, green and blue (RGB)) image frames of the same image capture area 310.

Each thermal image frame may then be sent by the thermal imaging device 124 to the controller 102 where it is received for processing by thermal imaging module 113 in memory 110.

At step 515, the thermal image frame is first processed by the thermal image processing module 113 to analyses the frame and locate human FoF areas within the image 400. The capability to ascertain FoF areas within the frame may be performed by face-detection algorithms stored within thermal image processing module 113. The face-detection algorithms may utilise a pre-trained machine learning model designed to detect human FoF areas within a thermal image frame 400.

Once all FoF areas have been isolated at 520, the identified faces are isolated into separate FoF frames. In some embodiments, individual FoF frames are generated for each face identified by the thermal image processing module 113.

The thermal image processing module 113 may also perform an exclusion process as part of the isolation process at 520. The exclusion process assessing the relative pixel size of the faces located within the FoF frame(s). The relative pixel size may be determined by taking the pixel size of the primary user's face 406 and using it as a relative function to determine which other faces should be retained and analysed in the subsequent process.

Faces found to be below a set threshold may be determined to be too far from the station 101 to be accurately assessed for illness symptoms, and/or assumed to not be travelling with the user 305 that is using the station 101. As such, it is beneficial to analyse both the user 305 of the self-service device, and entirety of the image capture area 310, which could also capture the FoF thermal data of travelling companions of the user 305, thereby allowing more persons to be assessed for illness symptoms. The determination of distance may also be made by or in combination with the output of the proximity sensor 160.

The isolation process conducted by thermal image processing module 113 may also exclude persons that are captured in the background of the image capture area 310. This may help to ensure that no alert is accidentally raised for a person that is not in the current travelling party using the self-service station 101.

FoF frames identified as belonging to unrelated persons to the travelling party may be discarded and not processed any further. In some embodiments, these frames are retained in memory 110, or sent over the network 140 to database 155 for storage, and/or FoF machine learning training.

The thermal image processing module 113 may then perform a symptom matching process for each captured FoF frame, at step 525. The matching process compares the thermal temperature data extracted from the thermal image frame 400 to known thermal profiles relating to symptoms of at least one illness. The thermal profiles may be stored in the memory 110 of self-service station 101. In other embodiments, the thermal profiles are stored within the thermal image processing module 113, client device memory 147, or within database 155. This matching process may be performed for each available FoF frame awaiting processing. The thermal image processing module 113 may store a set of heuristic data, related to the thermal profiles, that has been configured with parameters designed to identify symptoms of illness related to temperature of the FoF area of a person. At this step, the determination of respiratory rate made by the thermal image processing module 113 may be made for an identified person to be used in symptom matching.

In some embodiments, the thermal image processing module 113 stores thermal profiles in a row/column database format. In such embodiments, the following data is stored:

Symptom name—Comprising a readable name of the symptom that is being attempted to be detected by the invention;

Max Temperature—Comprising a two-decimal number that defines the high value in Celsius of the symptom match range that the detected temperature should be below in order to trigger a symptom match;

Min Temperature—Comprising a two-decimal number that defines the low values in Celsius of the symptom match range that the detected temperature should be above in order to trigger a symptom match;

Max Margin—Comprising a two-decimal number that defines a margin window above the Max Temperature that will also trigger a symptom match. This may be used for adjusting the symptom match parameters over time as new knowledge is gained.

Min Margin—Comprising a two-decimal number that defines a margin window below the Min Temperature that will also trigger a symptom match. This may be used for adjusting the symptom match parameters over time as new knowledge is gained.

In some embodiments, the symptom profiles may also comprise a respiratory rate range or datum to assist in determining a symptom match. The respiratory rate may comprise a measurement of breaths taken for minute, or another appropriate time interval. The measured or calculated respiratory rate may be used as an indicator to determine if a human is breathing at or within an expected rate range or outside the expected rate range. An abnormally low or high respiratory rate of breaths per minute may indicate that the person might be unwell or have some form of condition impacting their breathing ability. Accordingly, a breathing rate determined by controller 102 to be above a predetermined upper threshold or below a predetermined lower threshold may trigger controller 102 to determine a symptom match (or a likely symptom match), either alone or in combination with thermal profile data.

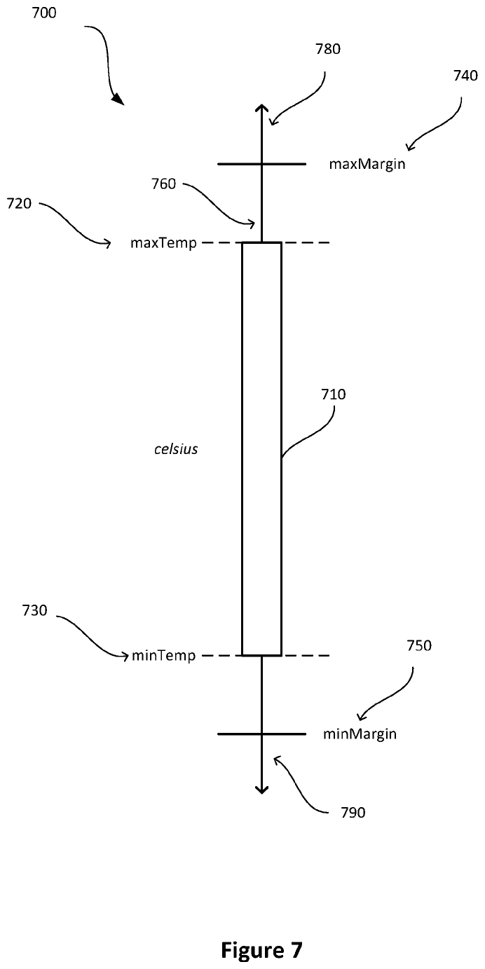

The symptom matching process undertaken at 525 may take the average temperature value in Celsius of the selected portion of a FoF frame, and compare it to the temperature values and ranges as illustrated in FIG. 7.

FIG. 7 depicts an example temperature profile range 700. Reference points 710, 760 and 770 define the range in which a symptom match will be triggered at step 535 if the temperature value is found to be within that range. Any temperature on the FoF frame found to be above the minTemp 730 minus the minMargin 750, or below the maxTemp 720 plus the maxMargin 740 will trigger a symptom match. This may include any temperature found to be in the margin area 760 above the maxTemp 720, or margin area 770 below the minTemp 730. Any temperature above the maximum range 780 or below the minimum range 790 may not trigger a symptom match. In some embodiments, each individual pixel of a FoF frame is compared against the profile range 700. In some embodiments, select groupings of pixels of FoF frames are compared against the profile range 700. In other embodiments, an average temperature of a grouping of pixels of FoF frames may be compared against the profile range 700.

In some embodiments, the thermal image processing module 113 may receive input from environmental sensor 135 at 535. The environmental sensor 135 may capture environmental data of the area around the self-service station 101. The environmental data may comprise temperature, humidity, pollution, or other environmental readings. The processor 105 may then send the captured environmental data back to thermal image processing module 113. The environmental data may be then used by thermal image processing module 113 in evaluation of a symptom match. In some embodiments, temperature data from the environmental sensor 135 provides a baseline ambient temperature, which may be subtracted from the temperature readings of pixels within a FoF frame. The environmental data may be stored within memory 110. In some embodiments, the environmental data is packaged by the communication module 111 and sent by wireless communication device 115 to be stored in database 155 over network 140.

In other embodiments, the environmental data may be used by the thermal image processing module 113 to modify the maximum or minimum levels of thermal profiles relating to at least one illness. In other embodiments, the thermal image processing module 113 may use the data to modify the maximum or minimum margin levels of a thermal profile.

The comparison of FoF frames to temperature profiles continues within thermal image processing module 113 at step 530 until there are no FoF frames left to process. Once all FoF frames have been isolated and have been thermally processed by thermal imaging module 113, the thermal image processing module 113 then determines whether the symptom matches a thermal profile at step 535. If a match occurs, the interaction process is suspended at step 550. In some embodiments, this is an immediate suspension, triggered at thermal image processing module 113, and sent to the user interface module 112 preventing the user from progressing the interaction process. In some embodiments, an instruction is shown to the user 305 on the user interface 120, indicating that they are required to wait for assistance from a staff member or station operator. In some embodiments, processor 105 is further configured to suspend the interaction process, at step 550, based on the determined respiratory rate.

At this point, once the interaction process is suspended, an alert message may be sent to the appropriate operator, generated by processor 105 which packages and sends the alert via the wireless communication device 115 as a data packet or object. The alert may be sent over network 140 to client device 145 and displayed on the client device 145 by application 148, for example.

The alert may contain details of the reason the alert has been generated, with information required to assist the operator when the operator approaches. This may include, but is not limited to, the location or number of the self-service station 101 reporting the alert, the user name, booking reference number and symptom heuristic that was matched by the station 101, for example.

In some embodiments, RGB image frames captured by image capture device 130 at step 510 are used to associate the FoF temperature data of a thermal image frame with an RGB image of the corresponding person. In such embodiments, the thermal image processing module 113 allocates a coordinate position to a FoF frame. The coordinate position of the FoF frame is then mapped to the RGB image frame captured by image capture device 130 to allow an RGB FoF frame of the thermally identified person to be produced. In such embodiments, any person having a FoF frame that triggers a symptom match may be associated with the RGB FoF frame by thermal image processing module 113. The RGB FoF frames of identified persons having symptom matches may then be attached to alerts issued by thermal image processing module 113. The attached RGB FoF frames, when received over network 140 by application 148 at client device 145, may assist an operator of client device 145 in identifying the person or persons triggering the match when inspected at step 610.

The alert issued by thermal imaging module 113 may be received by the operator at step 555 on a network-connected client device 145. The operator can view the alert information and then approach the person or persons using the self-service device that has sent the alert. The operator may then perform manual validation at 610 as deemed appropriate by standard operating procedures.

In some embodiments, the alert message is sent to the appropriate operator via a messaging system process between station 101 and the application 148 of client device 145. The messaging system comprises executable program code within application 148. In such embodiments, the application 148 may be configured to send and receive data via client device 145 over network 140. In some embodiments, the application 148 is configured to allow an operator to access or modify data stored in memory 110 of station 101, or database 155.

In some embodiments, the operator of client device 145 is logged into the application 148 using an authenticated credential to access the messaging system within application 148. In such embodiments, the operator specifies in the messaging system the physical location that the operator may be assigned to and working in. In some embodiments, this location is an airport, boarding gate, immigration gate, or other physical location or area. When an alert is sent by thermal image processing module 113, processor 105 of station 101 may retrieve a list of the staff members currently assigned to the area wherein the symptom match was found from memory 147, memory 110 over network 140, or database 155. In such embodiments, the thermal image processing module 113 directs communication module 111 to only send the alert to those appropriate operators based on the retrieved list.

In other embodiments, the retrieval of the staff member list and staff assignment is undertaken by application 148 when an alert is received there from station 101.

When an alert is created, the processor 105 may store the alert and current status in memory 110. The processor 105 may periodically check the status of the current alert by querying the memory 110 for the latest status. In some embodiments, the processor 105 may package the alert and send it for storage in database 150 as a data package or object.

At step 610, an operator is directed through application 148 to perform a manual validation step, checking whether the thermal image data of the relevant person have properly identified that operator intervention is required. In such embodiments, when an operator of the client device 145 indicates on application 148 whether no further action is required or further intervention is needed, the operator may indicate on the client device 145 to either ignore or accept the alert. The server 150 may update or modify the recorded status of the alert on database 155, which may then be communicated to the station 101 or client device 145 the next time that it queries the database 155 for the latest status. All alerts and actions on those alerts are stored in an auditable format in database 155, allowing for historic querying and reporting. Stored alert and action data includes data of the alert content, trigger conditions and outcome of action by the operator, including the staff number or other identifier of the operator that reviewed and actioned the alert. Database 155 can also store the FoF frame that triggered the symptom match to be used for later analysis and reporting.

If the manual validation of an operator at step 610 finds that the person is not diagnosed with (or suspected of) the relevant illness, then the operator may interact with the self-service station 101 or use client device 145 to communicate to the station 101 that the alert can be ignored and the user 305 can continue their check-in process as normal. At this point, the self-service station 101 may receive instructions at step 615 from the operator via client device 145, indicating whether the validation has passed. If the validation is passed, the user interface 120 may be accessed by the user 305 to complete the interaction process at step 620, using the self-service station 101 until the process has ended at step 625. If the validation has not passed, at step 616 the user initiated process at self-service station 101 is terminated.

The operation process 500 may not continue again until a new request for an interaction process is started by a new user initiating the process at self-service kiosk.

If the validation of step 610 is passed, and a user 305 is allowed to continue the transaction process, then remaining FoF frames for processing may be discarded. The frames may be discarded to prevent the interaction process at self-service station 101 being stopped for every subsequent frame and require overriding as each subsequent FoF frame is captured and processed.

If the manual validation process of step 610 finds that the person is diagnosed or suspected to be carrying a relevant illness, then the operator may interact with the self-service station 101 or use client device 145 to indicate that the process should be prematurely ended at step 616.

If the symptom matching process undertaken by thermal image processing module 113 finds no match between the FoF frames and the thermal profiles during the process of matching of step 535, then the thermal image processing module 113 may automatically move to processing the next available FoF frame. The thermal image processing module 113 may continue to process every available FoF frame for as long as the interaction process continues, until a FoF frame is found to have a symptom match or until the passenger processing application finishes as part of the normal operation. At this point, the thermal image processing module 113 may stop generating thermal image data and processing FoF frames until the next passenger transaction is started.

At step 630, the process 500 concludes. The self-service station 101 then becomes ready to receive a new interaction process request at user interface 120.

FIG. 8 is a flowchart 800 of a further method of operation of the interaction station according to some embodiments. In such embodiments, a heart rate monitoring system is provided as part of station 101 to provide additional data for the thermal imaging module 113 to conduct symptom match assessment. The heart rate monitoring system may be implemented by the processor 105 when executing program code within thermal image processing module 113. The heart rate monitoring system may be configured to monitor heart rate based on changes over time in images of faces identified in thermal and/or RGB images.

The user approaches the self-service station 101 and uses the user interface 120 to initiate an interaction process as normal. This action comprises the process of step 505 as described in relation to FIG. 5. The initiating request may involve following the instructions presented on the touch screen 122 in order to retrieve a booking and begin the interaction process.

At step 810, the operations of temperature analysis (as in FIG. 5) and heart rate analysis may begin in parallel with the user conducted interaction process. In such embodiments, the output of the heart rate analysis and detection process becomes another input to the symptom match step 535 and alerting function triggered in step 555.

The heart rate analysis process 800 is performed over a sample time period which allows for sufficient subject heart beats to be detected in order to form an observed heart rate, with the units of beats-per-minute (BPM). The sample time period may be 5 or 10 seconds allowing for enough sample heart beats to be observed in order to extrapolate and infer the expected number of beats per minute. In some embodiments, the sample time period is 1 to 2 seconds. In some embodiments, the sample time period may be a dynamic range, or user-specified range depending on thermal imaging module 113 requirements.

The heart rate analysis process 800 is performed by capturing and analysing two data inputs that are received from the thermal imaging device 125 and the image capture device 130. Thermal imaging device 125 is configured to provide thermal images as one input, and image capture device 130 is configured to provide RGB images as the other input.

At step 815, the thermal imaging device 125 provides thermal image frames to the thermal imaging module 113. The thermal images may comprise the same images as those captured at step 510 of FIG. 5. In other embodiments, a separate image capture process is undertaken by thermal imaging device 125. In some embodiments, a series of thermal image frames are sent to thermal image processing module 113. The thermal processing module 113 is configured to process the thermal images in order to capture acute temperature changes under the surface of the skin of a subject in the image capture area 310. At step 820, the thermal image processing module 113 processes the thermal image frames to identify a heart rate reading in BPM using the acute subdermal thermal changes identified in the thermal image frames.

At step 825, the image capture device 130 may provide RGB channel image frames to the thermal imaging module. In some embodiments, the image capture device 130 may comprise a colour camera positioned to capture RBG images of the image capture area 310 simultaneously with capture of the thermal images. In some embodiments, a series of RGB image frames are sent to thermal image processing module 113. The image capture device 130 is configured to capture acute brightness and colour changes on the surface of the skin of a subject in the image capture area 310. At step 830, thermal image processing module 113 (or a separate colour image processing module, not shown) processes the RGB image frames in order to identify a heart rate reading from the brightness and colour changes to skin identified in the RGB image frames.

In some embodiments, facial recognition module 114 is used at steps 815 and 825 on the image frames in order to isolate specific FoF frames for processing by thermal image processing module 113 at steps 820 and 830 respectively.

The heart rate readings obtained at step 820 and step 830 form two signals of a determined heart rate, relating to blood pulse. At step 835, the thermal image processing module 113 combines the two signals and processes them to create a data output for the sample time period referred to as observed heart rate, or determined heart rate. The process of combining thermal image heart rate data and RGB image heart rate data may be substantially similar to heart rate sensing techniques used in commercially available systems, such as the Microsoft Kinect 2™, or other similar devices, for example.

The determined heart rate becomes another symptom that can be checked against illness-related profiles stored in the memory 110. In some embodiments, the illness-related profiles may be stored in memory 110, database 155, or client device memory 147.

The capability to identify an observed heart rate may allow for symptom matching at step 840 to be more accurate and reduce false positives. This capability may allow for greater identification of symptoms of illnesses which may be observed in sufferers of communicable illnesses. The capability also allows for both these symptom matching processes to be performed without any physical contact to the self-service station 101, thereby reducing the potential for the self-service station 101 to become a surface for transmission of a contagious illness between users using the self-service station 101 in an airport, or similar environment, where control of contagions and hygiene are particularly sensitive.

At step 840, thermal image processing module 113 compares determined heart rate data with illness-related heart rate data. If a symptom match between the determined heart rate is established at step 840 and illness-related heart rate data, then the user interaction process is suspended at 845. If a symptom match is not established at step 840, and the user interaction process has yet to conclude, the process 800 reverts to step 810. The process 800 may take place in a continuous loop for the duration of the user interaction in order to continuously observe the subject person's heart rate.

If a symptom match is identified at step 840, the processor 105 follows the process as per step 550 in FIG. 5, beginning with suspending the passenger processing application transaction 550 and alerting an operator for manual verification at step 555.

If no symptom match is identified, the process 800 continues the heart rate analysis for the next sample time period and performs the process 800 over again until a symptom match is found or the interaction process at station 101 ends. If, at step 835, there is no symptom match identified, and the interaction process of the user 305 has concluded, then the process 800 is concluded at 850.

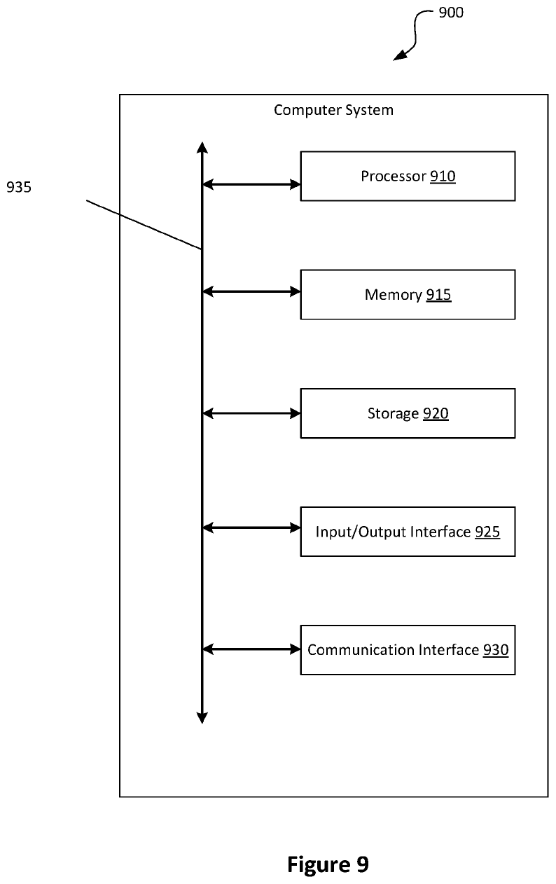

FIG. 9 illustrates an example computer system 900 according to some embodiments. In particular embodiments, one or more computer systems 900 perform one or more steps of one or more methods described or illustrated herein. In particular embodiments, one or more computer systems 900 provide functionality described or illustrated herein. In particular embodiments, software running on one or more computer systems 900 performs one or more steps of one or more methods described or illustrated herein or provides functionality described or illustrated herein. Particular embodiments include one or more portions of one or more computer systems 900. Herein, reference to a computer system may encompass a computing device, and vice versa, where appropriate. Moreover, reference to a computer system may encompass one or more computer systems, where appropriate. Controller 102 is an example of computer system 900.

This disclosure contemplates any suitable number of computer systems 900. This disclosure contemplates computer system 900 taking any suitable physical form. As example and not by way of limitation, computer system 900 may be an embedded computer system, a system-on-chip (SOC), a single-board computer system (SBC) (such as, for example, a computer-on-module (COM) or system-on-module (SOM)), a special-purpose computing device, a desktop computer system, a laptop or notebook computer system, a mobile telephone, a server, a tablet computer system, or a combination of two or more of these. Where appropriate, computer system 900 may: include one or more computer systems 900; be unitary or distributed; span multiple locations; span multiple machines; span multiple data centers; or reside partly or wholly in a computing cloud, which may include one or more cloud computing components in one or more networks. Where appropriate, one or more computer systems 900 may perform without substantial spatial or temporal limitation one or more steps of one or more methods described or illustrated herein. As an example and not by way of limitation, one or more computer systems 900 may perform in real time or in batch mode one or more steps of one or more methods described or illustrated herein. One or more computer systems 900 may perform at different times or at different locations one or more steps of one or more methods described or illustrated herein, where appropriate.

In particular embodiments, computer system 900 includes at least one processor 910, memory 915, storage 920, an input/output (I/O) interface 925, a communication interface 930, and a bus 935. Processor 105 is an example of processor 910. Memory 110 is an example of memory 915. Memory 110 may also be an example of storage 920. Although this disclosure describes and illustrates a particular computer system having a particular number of particular components in a particular arrangement, this disclosure contemplates any suitable computer system having any suitable number of any suitable components in any suitable arrangement.

In particular embodiments, processor 910 includes hardware for executing instructions, such as those making up a computer program. As an example and not by way of limitation, to execute instructions, processor 910 may retrieve (or fetch) the instructions from an internal register, an internal cache, memory 915, or storage 920; decode and execute them; and then write one or more results to an internal register, an internal cache, memory 915, or storage 920. In particular embodiments, processor 910 may include one or more internal caches for data, instructions, or addresses. This disclosure contemplates processor 910 including any suitable number of any suitable internal caches, where appropriate. As an example and not by way of limitation, processor 910 may include one or more instruction caches, one or more data caches, and one or more translation lookaside buffers (TLBs). Instructions in the instruction caches may be copies of instructions in memory 915 or storage 920, and the instruction caches may speed up retrieval of those instructions by processor 910. Data in the data caches may be copies of data in memory 915 or storage 920 for instructions executing at processor 910 to operate on; the results of previous instructions executed at processor 910 for access by subsequent instructions executing at processor 910 or for writing to memory 915 or storage 920; or other suitable data. The data caches may speed up read or write operations by processor 910. The TLBs may speed up virtual-address translation for processor 910. In particular embodiments, processor 910 may include one or more internal registers for data, instructions, or addresses. This disclosure contemplates processor 910 including any suitable number of any suitable internal registers, where appropriate. Where appropriate, processor 910 may include one or more arithmetic logic units (ALUs); be a multi-core processor; or include one or more processors 910. Although this disclosure describes and illustrates a particular processor, this disclosure contemplates any suitable processor.

In particular embodiments, memory 915 includes main memory for storing instructions for processor 910 to execute or data for processor 910 to operate on. As an example and not by way of limitation, computer system 900 may load instructions from storage 920 or another source (such as, for example, another computer system 900) to memory 915. Processor 910 may then load the instructions from memory 915 to an internal register or internal cache. To execute the instructions, processor 910 may retrieve the instructions from the internal register or internal cache and decode them. During or after execution of the instructions, processor 910 may write one or more results (which may be intermediate or final results) to the internal register or internal cache. Processor 910 may then write one or more of those results to memory 915. In particular embodiments, processor 910 executes only instructions in one or more internal registers or internal caches or in memory 915 (as opposed to storage 920 or elsewhere) and operates only on data in one or more internal registers or internal caches or in memory 915 (as opposed to storage 920 or elsewhere). One or more memory buses (which may each include an address bus and a data bus) may couple processor 910 to memory 915. Bus 935 may include one or more memory buses, as described below. In particular embodiments, one or more memory management units (MMUs) reside between processor 910 and memory 915 and facilitate accesses to memory 915 requested by processor 910. In particular embodiments, memory 915 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 915 may include one or more memories 915, where appropriate. Although this disclosure describes and illustrates particular memory, this disclosure contemplates any suitable memory.

In particular embodiments, storage 920 includes mass storage for data or instructions. As an example and not by way of limitation, storage 920 may include a hard disk drive (HDD), a floppy disk drive, flash memory, an optical disc, a magnetooptical disc, magnetic tape, or a Universal Serial Bus (USB) drive or a combination of two or more of these. Storage 920 may include removable or non-removable (or fixed) media, where appropriate. Storage 920 may be internal or external to computer system 900, where appropriate. In particular embodiments, storage 920 is non-volatile, solid-state memory. In particular embodiments, storage 920 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 920 taking any suitable physical form. Storage 920 may include one or more storage control units facilitating communication between processor 910 and storage 920, where appropriate. Where appropriate, storage 920 may include one or more storages 920.

Although this disclosure describes and illustrates particular storage, this disclosure contemplates any suitable storage. In particular embodiments, I/O interface 925 includes hardware, software, or both, providing one or more interfaces for communication between computer system 900 and one or more I/O devices. Computer system 900 may include one or more of these I/O devices, where appropriate. One or more of these I/O devices may enable communication between a person and computer system 900. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 925 for them. Where appropriate, I/O interface 925 may include one or more device or software drivers enabling processor 910 to drive one or more of these I/O devices. I/O interface 925 may include one or more I/O interfaces 925, where appropriate. Although this disclosure describes and illustrates a particular I/O interface, this disclosure contemplates any suitable I/O interface.

In particular embodiments, communication interface 930 includes hardware, software, or both providing one or more interfaces for communication (such as, for example, packet-based communication) between computer system 900 and one or more other computer systems 900 or one or more networks. As an example and not by way of limitation, communication interface 930 may include a network interface controller (NIC) or network adapter for communicating with a wireless adapter for communicating with a wireless network, such as a WI-FI or a cellular network. This disclosure contemplates any suitable network and any suitable communication interface 930 for it. As an example and not by way of limitation, computer system 900 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 900 may communicate with a wireless cellular telephone network (such as, for example, a Global System for Mobile Communications (GSM) network, or a 3G, 4G or 5G cellular network), or other suitable wireless network or a combination of two or more of these. Computer system 900 may include any suitable communication interface 930 for any of these networks, where appropriate. Communication interface 930 may include one or more communication interfaces 930, where appropriate. Although this disclosure describes and illustrates a particular communication interface, this disclosure contemplates any suitable communication interface.

In particular embodiments, bus 935 includes hardware, software, or both coupling components of computer system 900 to each other. As an example and not by way of limitation, bus 935 may include an Accelerated Graphics Port (AGP) or other graphics bus, an Enhanced Industry Standard Architecture (EISA) bus, a frontside bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 935 may include one or more buses 935, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, (FDDs), solid-state drives (SSDs), RAM-drives, or any other suitable computer-readable non-transitory storage media, or any suitable combination of two or more of these, where appropriate. A computer-readable non-transitory storage medium may be volatile, non-volatile, or a combination of volatile and non-volatile, where appropriate.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A self-service station for conducting an interaction process, comprising:
a memory storing executable program code;
a processor in communication with the memory, the processor configured to execute the program code stored within the memory;
a user interface in communication with the processor, the user interface configured to allow a user to initiate the interaction process;
a thermal imaging device in communication with the processor, the thermal imaging device configured to capture thermal images of an area from which the user interface is accessible;
wherein the processor is configured to:
process thermal images captured by the thermal imaging device;
determine a temperature condition of a face identified in at least one of the captured thermal images; and
suspend the interaction process based on the determined temperature condition.

2. The station of claim 1, wherein the processor is further configured to identify front-of-face thermal data, based on the thermal images.

3. The station of claim 1 or claim 2, further including a data store, wherein the processor is further configured to compare temperature data derived from the thermal images to illness-related temperature data stored in the data store and to suspend the interaction process when the temperature data matches illness-related temperature data.

4. The station of claim 3, wherein the data store comprises a repository of temperature data profiles associated with symptoms of at least one illness.

5. The station of any one of claims 1 to 4, wherein the interaction process is a multi-step process that can take between about 1 minute and about 20 minutes to complete when not suspended.

6. The station of any one of claims 1 to 5, wherein the processor is configured to determine heart rate data based on changes over time in images of the face identified in the at least one of the thermal images.

7. The station of any one of claims 1 to 6, further comprising a colour camera positioned to capture RGB images of the area simultaneously with capture of the thermal images.

8. The station of claim 7, wherein the processor is configured to receive captured RGB images from the colour camera and to determine heart rate data based on changes over time in images of the face identified in the captured RGB images.

9. The station of claim 8, wherein the processor is configured to suspend the interaction process based on the determined temperature condition and the determined heart rate data.

10. The station of any one of claims 6 to 9, wherein the processor is configured to compare the determined heart rate data to stored illness-related heart rate data accessible to the processor.

11. The station of any one of claims 1 to 10, wherein the processor is further configured to generate an alert based on the determined temperature condition.

12. The station of claim 11, wherein the processor is configured to send the alert via a communication module of the station to a server or a client device over a network that is accessible to the communication module.

13. The station of claim 11 or claim 12, wherein the alert includes a unique identifier of the station and at least one of: an image of the face; the determined temperature condition; user identification information received via the user interface; illness-related information associated with the determined temperature condition; or user booking information retrieved from a data store based on user identification information received via the user interface.

14. The station of any one of claims 1 to 13, wherein the processor is configured to use a trained machine learning model to identify the face in at least one of the captured thermal images.

15. The station of any one of claims 1 to 14, wherein the processor is configured to identify multiple faces in at least one of the captured thermal images and to determine which of one or more of the multiple faces are proximate to the station.

16. The station of claim 15, wherein the processor is configured to determine the temperature condition of each of the multiple faces and to suspend the interaction process when the temperature condition of at least one of the multiple faces matches a temperature data profile associated with symptoms of at least one illness.

17. A system for conducting self-service interaction processes, the system including:
at least one self-service station according to any one of claims 1 to 16; and
at least one client computing device in communication with the at least one self-service station;
wherein the processor of each at least one self-service station is configured to resume the interaction process in response to receiving a resume message from the at least one client computing device.

18. The system of claim 17, further including a server in communication with the at least one self-service station and the at least one client computing device.

19. The system of claim 18, wherein communication between the at least one self-service station and the at least one client computing device is routed through the server.

20. A computer-implemented method of conducting an interaction process at a self-service station comprising the steps of:
receiving an initiation request at a user interface to initiate an interaction process;
capturing a thermal image with a thermal imaging device;
processing a thermal image to determining whether temperature data based on the thermal image matches a stored profile; and
suspending the transaction process when it is determined that the temperature data matches a stored profile.

21. A system for conducting an interaction process, comprising:
at least one of the self-service station of any one of claims 1 to 16, wherein the at least one self-service station is configured to transmit alert data over a network if the interaction process is suspended;
a client device configured to receive alert data from the self-service station over the network.

22. The system of claim 21, wherein the client device is configured to send a message to the station to cancel or resume the interaction process.

23. The station of any one of claims 1 to 16, further comprising a proximity sensor in communication with the processor, the proximity sensor being configured to determine a proximity measurement in the area from which the user interface is accessible.

24. The station of claim 23, wherein the processor is configured to suspend the interaction process based on the determined proximity measurement.

25. The station of any one of claims 1 to 16, 23 and 24, wherein the processor is further configured to determine a respiratory rate based on thermal images of the face identified in captured thermal images received over a time interval.

26. The station of claim 25, wherein the processor is further configured to suspend the interaction process based on the determined respiratory rate.

27. A system for conducting self-service interaction processes, the system including:
at least one self-service station according to any one of claims 23 to 26; and
at least one client computing device in communication with the at least one self-service station;
wherein the processor of each at least one self-service station is configured to resume the interaction process in response to receiving a resume message from the at least one client computing device.

28. A system for conducting an interaction process, comprising:
at least one of the self-service station of any one of claims 23 to 26, wherein the at least one self-service station is configured to transmit alert data over a network if the interaction process is suspended;
a client device configured to receive alert data from the self-service station over the network.

29. The steps, systems, devices, subsystems, features, integers, methods and/or processes disclosed herein or indicated in the specification of this application individually or collectively, and any and all combinations of two or more of said steps, systems, devices, subsystems, features, integers, methods and/or processes.
